(19) United States
(12) Patent Application Publication
(10) Pub. No.: US 2021/0204878 A1
(43) Pub. Date: Jul. 8, 2021

(54) PERCUTANEOUS MICRONEEDLE MONITORING SYSTEM

(71) Applicant: Jung-Tang Huang, Taipei (TW)

(72) Inventor: Jung-Tang Huang, Taipei (TW)

(73) Assignee: Jung-Tang Huang, Taipei (TW)

(21) Appl. No.: 16/950,514

(22) Filed: Nov. 17, 2020

(30) Foreign Application Priority Data
Nov. 19, 2019 (TW) 108141981

(51) Int. Cl. A61B 5/00; A61B 5/1473; A61B 5/145; A61B 5/1486

(52) U.S. Cl. CPC A61B 5/685; A61B 5/1473; A61B 5/14514; A61B 5/14865; A61B 5/14532; A61B 5/14546; A61B 5/411; A61B 5/4833

(57) ABSTRACT

A percutaneous microneedle monitoring system is provided with a substrate, a microneedle unit, a signal processing unit and a power supply unit, wherein the microneedle unit is created by stacking a plurality of metal sheets with protruding microneedle arrays on the substrate. Each sheet is provided with at least one perforation and the perforation edge is provided with a spur. Perforation on one sheet allows the spurs of the perforation edges to pass through at opposite positions on the remaining sheets, and the spurs are separated from each other. The microneedle unit is equipped with a signal processing unit to continuously detect the concentration changes of the various analytes appearing in the tissue fluid by fixing sensing polymer on the inner surface of protruding spur of the microneedle unit.

### BACKGROUND OF THE INVENTION

### 1. Field of the Invention

The present invention concerns a percutaneous microneedle monitoring system, in particular a percutaneous microneedle array, which can measure the analytes found in the dermis interstitial fluid (ISF) and measure the concentration of target drug molecules under the skin, thus recognizes the compliance of medications and the pharmacokinetics of animal body.

### 2. Description of Related Art

The advancement of technologies that can continuously and in real time control the concentration of drugs, metabolites and biomarkers in the body can enhance human health sensitivity and the ability to diagnose and treat diseases. For example, it will allow high-resolution, patient-specific pharmacokinetics (including feedback-controlled drug delivery) treatments, creating a new area of personalized medicine.

When taking or administering drugs, the drugs will be released in the tissue fluid for a long time and slowly. During the clinical trials of drug development and use, it is often necessary to constantly monitor the concentration changes of the drug in the tissue fluid, and therefore sampling the tissue fluid for testing or analysis can be seen everywhere in medical procedures. To monitor medication compliance, it is necessary not only to know whether to take the medicine, but also to confirm the time to take the right medicine. In addition, the better way to take the medicine is to adjust it according to your physical condition and whether the symptoms described in the topic have changed, for example, a medicine that lowers your heartbeat. If your heartbeat has decreased or returned to normal for other reasons, you may have to stop or reduce the dose. The same is true for medicines that lower blood pressure. Therefore, it is highly necessary to know the real-time changes of the drug concentration in the blood.

Currently, therapeutic drug monitoring (TDM) relies on concentration measurement performed in large medical laboratories and requires the collection of milliliters of blood samples, which may be uncomfortable for long-term treatment patients. The specimen needs to be transferred to a professional laboratory for measurement. After the inspection by the laboratory staff, the measurement results are sent to the doctor in charge of the patient. The clinical interpretation of the measurement results represents a key challenge, as doctors need to make decisions about translating drug concentration values into appropriate dose adjustments. It may take several hours in total in non-emergency situations, which has potential adverse effects on patients in critical conditions. Therefore, it is very important to know the real-time changes of the drug concentration in the blood simply, quickly and conveniently.

Next, in terms of the subcutaneous tissue, the subcutaneous tissue is the main place where human tissue fluid flows and distributes. The tissue fluid is rich in amino acids, sugars, fatty acids, coenzymes, hormones, neurotransmitters, salts, waste products produced by cells, and drugs, etc. The tissue fluids are the main channels through which cells communicate with blood. Therefore, the concentration of each component in the tissue fluid is one of the methods used to judge physiological conditions.

The literature in the past shows that ordinary beverages contain three very similar compounds-all in the same group of alkaloids, namely caffeine, theophylline and theobromine. The subject, after a known method, mentioned enough interstitial fluid (ISF) is measured. For example, the highest average blood glucose concentration detected 1 h after ingestion of 75 g of glucose is 7.89 nmol/L, and the highest average glucose concentration detected 3 hours after insertion from the skin of a human is 4.29 nmol/L.

However, the stratum corneum, which is the outermost layer of the skin, has evolved into an effective barrier to the outward migration of body fluids, and appropriate techniques are required to extract a sufficient amount of ISF for analysis. The conventional technology has proposed reverse iontophoresis (RI) and clinical microdialysis (CM) as a means of using ISF in percutaneous monitoring. However, in reverse iontophoresis, large, complicated and expensive equipment is usually required, and professional operation is required. In addition, due to the net negative charge of the skin, several hours of lag time and patient sweating may delay and impair accuracy, anions cannot be extracted in significant amounts. Similarly, in clinical microdialysis, the probe is often difficult to locate and must be completed by appropriately trained medical personnel. In addition, tissue damage at the probe insertion site often affects the accuracy of the measurement.

To sum up, most of the physiological testing equipment on the market today, or the method for medical staff to sample tissue fluid, use needles and puncture the stratum corneum to extract tissue fluid for analysis and testing. However, this type of sampling method that destroys the skin surface in addition to making the patient feel painful, will lead to rejection, a large number of microorganisms on the skin surface are also easy to enter the animal body and become infected when the skin surface is destroyed. In order to improve the shortcomings of needle sticking and piercing the stratum corneum for sampling, a percutaneous sensor is proposed, which uses arrayed microneedles for skin puncture. The low-invasive puncture can effectively reduce the user's pain and achieve the purpose of tissue fluid sampling at the same time.

The microneedle array sensor, like other in-vivo biosensors, will gradually become a powerful tool in biomedical research and diagnostic medicine. Different from “tagging” or “imaging”, in vivo biosensors are designed for continuous and long-term monitoring of target analytes in actual biological systems, so they must have selectivity, sensitivity, reversibility, and biocompatibility. Due to the challenges associated with meeting all analytical requirements, relatively few research reports have shown that the device can meet these stringent requirements.

At present, silicon wafers or electroforming are used to make molds, and macromolecules, especially hydrogels, are used to make microneedle arrays. An attempt is made to puncture the subcutaneous dermis, using hydrogels to swell with water. The porous characteristic of hydrogel can absorb tissue fluid, and then take out the microneedle array that is full of tissue fluid, and then extract tissue fluid for analysis. This method is batch-based, cannot detect in real time, and has complicated procedures.

The main detection target of the continuous percutaneous microneedle sensor currently on the market is blood glucose. The technology adopted is to use a soft electronic method to fabricate three electrochemical electrodes on a single microneedle. The specificity of the measurement is achieved by glucozyme. Since the blood glucose concentration is quite high in the blood, and other metabolites such as lactic acid and uric acid are only about 1/10 of the blood glucose concentration, the commercial CGMS products have not been extended. The measurement of the concentration of other metabolites, let alone the concentration of the drug in the blood, is only 1/1000 or lower of the blood glucose concentration. Moreover, the specific detection of drug molecules will not be achieved using enzymes.

From the above, it can be seen that for targets that cannot be detected by enzymes or analytes other than blood sugar, basically only blood can be drawn from the subject, and then non-continuous measurement using instruments or biochips in vitro. In addition, the commercially available continuous blood glucose monitoring system uses extremely fine microneedles (soft needles with a diameter of 200-300 microns), and three electrodes (working, auxiliary, and reference) are arranged on the same microneedle. The function of the working electrode area is very limited. Usually, even for blood glucose as high as 25 mg/dL or more, the current can only have an electrochemical reaction of nA level. Therefore, even the commercial CGMS passes the FDA, its performance lacks repeatability and credibility generally at a low blood glucose concentration of 50 mg/dL (0.5 mg/ml). As a result, the use of a single microneedle system like CGMS basically cannot achieve a lower concentration of reliable measurement, such as 0.1-100 ng/ml, which means that compared with the current CGMS, reliable measurement is required. The sensitivity needs to be increased thousands to tens of thousands of times to achieve the purpose of continuous measurement. Therefore, the present invention is to provide an innovative device and method to achieve reliable and continuous measurement of analytes (such as drugs, metabolites and biomarkers) contained in blood.

The fabrication of microneedles in transcutaneous sensors usually uses semiconductor processes such as lithography and etching. For example, the specification of U.S. Pat. No. 7,344,499 B1, column 12, paragraph 2, discloses a silicon microneedle process. First, a silicon wafer covered with a patterned first photoresist layer is provided. Then, etching is performed by isotropic etching to form a through hole. Then, a chromium layer is coated on the surface of the wafer, and then a patterned second photoresist layer is coated so as to cover the through holes, and a circular mask is formed for subsequent etching. Then, etching is performed to form the outer cone wall of the microneedle. However, due to the brittleness of the silicon-containing semiconductor material, when the microneedle punctures the skin for sensing, the microneedle is easily broken. Moreover, this patent does not propose a method that can truly continuously measure the concentration of the drug in the tissue fluid.

The invention previously applied by the inventor US2015208985, etc., using multiple microneedle arrays can improve the above-mentioned insufficient sensitivity, but its disadvantage is that the working electrode, counter electrode, and reference electrode are set at different positions. The area that the needle needs to contact with the skin becomes larger, and the applicable attachment site needs to be carefully selected. For example, the area with too large curvature may not be suitable; secondly, if the three electrodes are inserted into the subcutaneous depth at the same time, or the contact with the tissue fluid is not consistent, there is a possibility of inaccurate measurement and unreliability. Thirdly, they are arranged separately, and the strength is weak when assembled.

More specifically, the conventional sensing microneedle contacts the skin and partially invades the subcutaneously. During measurement, the working electrode and counter electrode or reference electrode of the microneedle patch are in contact with the skin respectively, resulting in the positive electrode and the negative electrode are separated by a certain distance. When the measurement method of the potentiostat is used, the sweat gland between the positive electrode and the negative electrode produces a reverse iontophoresis and ion penetration effect, which stimulates the discharge of sweat and affects the measurement of tissue fluid concentration of the analyte and its biochemical signal. In addition, the effect of reverse ion osmosis to stimulate sweat has also become one of the reasons for the failure of Glucowatch.

In view of the above-mentioned shortcomings, the present invention proposes an improved method to provide an all-in-one microneedle set and structure, so that three electrodes are arranged at the same position, which can simultaneously measure the concentration changes of more analytes in the tissue fluid and continuously measure the drug concentration in the tissue fluid.

### SUMMARY OF THE INVENTION

The objective of the present invention is to provide a percutaneous microneedle monitoring system. The microneedles of the microneedle group are formed by stamping or etching or electroforming processes, and have sufficient mechanical strength. During sensing, the microneedles can remain intact. Moreover, the structure of the working electrode microneedle assembly of the present invention is advantageous for coating the sensing polymer on the inner surface of the tip of the microneedle, which can reduce exfoliation of sensing polymer when the microneedles of the working electrode microneedle assembly pierce the skin for sensing.

The percutaneous microneedle monitoring system of the present invention includes: a substrate; a microneedle unit, at least including a first microneedle group and a second microneedle group arranged on the substrate, the first microneedle group is used as a working electrode, the second microneedle group is used as a reference electrode, and each microneedle group includes at least one microneedle and is a thin sheet. The first and second microneedle groups overlap each other but are electrically insulated from each other. Each sheet is provided with at least one perforation, and the perforation edge is provided with a spur. The perforation on one of the sheets allows the spurs of the perforation edges at opposite positions on the remaining sheets to pass through, and the spurs are separated from each other; a signal processing unit, which is arranged on the substrate and electrically connected to the first and second microneedle groups; and a power supply unit, which supplies working power to the monitoring system.

More specifically, the first microneedle group is formed by stacking a sheet and a sheet of the second microneedle group, but they are electrically insulated from each other. At least a first perforation, the edge of the first perforation is provided with a first spur, and the sheet of the second microneedle group is provided with at least one second perforation, the edge of the second perforation is provided with a second spur, and the second spur passes through the first perforation at a relative position on the sheet of the first microneedle group is opposite to the first spur.

More specifically, the microneedle unit further includes a third microneedle group as a counter electrode.

More specifically, the first microneedle group is formed by stacking a first sheet, a second microneedle group by a second sheet, and a third microneedle group by a third sheet, but they are mutually electrically insulated. The first sheet is provided with at least one first perforation, the first perforation edge is provided with a first spur, the second sheet is provided with at least one second perforation, the second perforation edge is provided with a second spur, and the at least one third perforation is provided on the third sheet, the edge of the third perforation is provided with a third spur, the second spur and the third spur pass through the first perforation on the first sheet and the first spur is triangular conical or quadrangular pyramid with missing one side.

More specifically, the microneedle unit further includes a fourth microneedle group as the second working electrode.

More specifically, the first microneedle group consists of a first sheet, a second microneedle group consists of a second sheet, a third microneedle group consists of a third sheet, and a fourth microneedle group consists of a fourth sheet. The first sheet is formed by stacking but electrically insulated from each other. The first sheet is provided with at least one first perforation, the edge of the first perforation is provided with a first spur, the second sheet is provided with at least one second perforation, the second The perforation edge is provided with a second piercing, the third sheet is provided with at least one third perforation, the third perforation edge is provided with a third perforation, and the fourth sheet is provided with at least a fourth perforation, and the fourth perforation edge is provided with a fourth spur. The second spur, the third spur and the fourth spur pass through the first perforation on the first sheet and form a quadrangular pyramid with the first spur.

More specifically, the further includes adding at least one microneedle unit, which can simultaneously sense the subcutaneous analyte or/and more types of drugs.

More specifically, the microneedles of the first microneedle group, the second microneedle group, the third microneedle group, and the fourth microneedle group are formed by a stamping or etching or electroforming process.

More specifically, the signal processing unit is mainly selected from electrochemical sensing circuits, amperometry, square wave voltammetry (SWV), differential pulse wave voltammetry (DPV), chronoamperometry, intermittent pulse amperometry (IPA), fast-scan cyclic voltammogram (FSCV), electrochemical impedance spectrum (EIS) or a combination.

More specifically, the working electrode further includes a porous protective layer formed on the sensing polymer or an anti-skin allergy drug.

More specifically, the material of the spurs is selected from stainless steel, nickel, nickel alloy, titanium, titanium alloy or silicon material, and a biocompatible metal is deposited on the surface.

More specifically, the material of the spikes is resin, and biocompatible metal or conductive material is deposited on the surface.

More specifically, the height of the spurs is 300-3000 microns.

More specifically, the base width of the spurs is 150-450 microns.

More specifically, the distance between the tips of the spurs is 500-3000 microns.

More specifically, the inner surface of the working electrode is modified with a sensing polymer, and the sensing polymer is an antibody, aptamer, recombinant monomer (ScFv), carbohydrate, which is specific for the target analyte, one end of the sensing polymer is modified with a self-assembled monolayer (SAM), which can be fixed on the inner surface of the working electrode.

More specifically, the inner surface of the working electrode is modified with a sensing polymer, and the sensing polymer is an enzyme specific for the target analyte.

More specifically, the inner surface of the working electrode is modified with a sensing polymer, which is an aptamer specific to the target drug molecule, and one end of which is modified with a self-assembled monolayer (SAM), which can be fixed on the inner surface of the working electrode, the other end is modified with redox reporter molecules.

The percutaneous microneedle monitoring system of the present invention includes: a signal processing device, which includes a signal processing unit, a power supply unit, a female connector, a cover plate and an outer cover, wherein the signal processing unit, the power supply unit and the female connector are arranged on a circuit board; a micro-needle device includes a substrate, a base, a micro-needle unit, a flexible adhesive cloth, a release paper, and a male connector. The needle unit and the male connector are arranged on the substrate, and the substrate is embedded in the base, and the microneedle unit at least includes a first microneedle group and a second microneedle group arranged on the substrate And a third microneedle group, the first microneedle group is used as a working electrode, the second microneedle group is used as a reference electrode, the third microneedle group is used as a counter electrode, and the inner surface of the working electrode is modified with a sensing polymer and A porous protective layer; and the electrical connection between the signal processing device and the microneedle device is achieved by a connector, the connector of the microneedle device is a male connector, and the connector of the signal processing device is a female connector, and vice versa However, in addition, the mechanical connection between the signal processing device and the microneedle device is achieved by the outer cover and the base.

More specifically, the signal processing unit receives the analyte concentration sensed by the microneedle unit, and after calculation and determination, it can be converted into a sensing signal, and further transmitted to a user's hand-held device via a wireless communication method to reflect the current physiological state signal of the user.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is an external view of the percutaneous microneedle monitoring system of an embodiment of the present invention.

FIG. 2 is an exploded view of the percutaneous microneedle monitoring system of an embodiment of the present invention.

FIG. 3 is an exploded view of the transcutaneous microneedle monitoring system of another embodiment of the present invention.

FIG. 4 is an exploded view of the signal processing device of one embodiment of the percutaneous microneedle monitoring system of the present invention.

FIG. 5A is an exploded view of the first embodiment structure of the microneedle unit of the percutaneous microneedle monitoring system of the present invention.

FIG. 5B is a perspective view of the first embodiment of the microneedle unit of the percutaneous microneedle monitoring system of the present invention.

FIG. 6A is a partial top view of the second embodiment structure of the microneedle unit of the percutaneous microneedle monitoring system of the present invention.

FIG. 6B is a perspective view of the second embodiment of the microneedle unit of the percutaneous microneedle monitoring system of the present invention.

FIG. 7A is a partial top view of the third embodiment of the microneedle unit of the percutaneous microneedle monitoring system of the present invention.

FIG. 7B is a perspective view of the third embodiment of the microneedle unit of the percutaneous microneedle monitoring system of the present invention.

FIG. 8 is a partial top view of the fourth embodiment of the microneedle unit of the percutaneous microneedle monitoring system of the present invention

FIG. 9A is an exploded view of the microneedle device of the percutaneous microneedle monitoring system of the present invention.

FIG. 9B is a schematic side view of the combined microneedle device of the percutaneous microneedle monitoring system of the present invention.

FIG. 10A is a schematic diagram of the fifth embodiment of the microneedle unit of the percutaneous microneedle monitoring system of the present invention.

FIG. 10B is a schematic diagram of the fifth embodiment of the microneedle unit of the percutaneous microneedle monitoring system of the present invention.

FIGS. 10C and 10D are both schematic diagrams of the fifth embodiment of the microneedle unit of the percutaneous microneedle monitoring system of the present invention.

FIG. 11A is a schematic diagram of the fifth embodiment of the microneedle unit of the percutaneous microneedle monitoring system of the present invention.

FIG. 11B-1 is a schematic diagram of the first perspiration blocking embodiment of the percutaneous microneedle monitoring system of the present invention.

FIG. 11B-2 is a schematic diagram of the first perspiration prevention application of the percutaneous microneedle monitoring system of the present invention.

FIG. 11C is a schematic diagram of the second perspiration blocking embodiment structure of the percutaneous microneedle monitoring system of the present invention.

FIG. 11D is a schematic diagram of the third perspiration blocking embodiment structure of the percutaneous microneedle monitoring system of the present invention.

### DESCRIPTION OF THE EMBODIMENTS

The following is a description of specific embodiments to describe the embodiment of the “percutaneous microneedle monitoring system” disclosed by the present invention. Those skilled in the art can understand the advantages and effects of the present invention from the content disclosed in this description. The present invention can be implemented or applied through other different specific embodiments, and various details in this specification can also be based on different viewpoints and applications, and various modifications and changes can be made without departing from the concept of the present invention. In addition, the drawings of the present invention are merely a schematic illustration, and are not drawn according to actual dimensions, and are stated in advance. The following embodiments will further describe the related technical content of the present invention in detail, but the disclosed content is not intended to limit the protection scope of the present invention.

It should be understood that although the terms first, second, third, etc. may be used herein to describe various elements or signals, these elements or signals should not be limited by these terms. These terms are mainly used to distinguish one element from another element, or a signal from another signal. In addition, the term “or” as used herein should, depending on the actual situation, include any one or more of the associated listed items.

In the percutaneous microneedle monitoring system of the present invention, the sensing polymers used in the percutaneous microneedle sensor basically include specific aptamers, antibodies, etc., wherein the universality of aptamers is derived from the multifunctional recognition and signal transduction properties of the aptamers, the ability of nucleic acids to be selected for binding to specific molecular targets. Through the use of sophisticated in vitro selection methods, aptamers that can bind to a wide range of analytes can be generated, and can be reasonably redesigned to cause large-scale conformational changes when they bind to these analytes within any wide or narrow concentration window. The percutaneous microneedle sensor uses this conformational change to generate an electrochemical signal that is easy to measure without the need for target chemical transformation. In order to achieve this signal transduction, the binding of the aptamer is used to induce conformational changes to change the efficiency of the covalently linked redox reporter molecule (here, methylene blue) close to the underlying electrode, which produces a target concentration dependence on the change in current when the sensing electrode is interrogated by square wave voltammetry. In accordance with the requirements to support continuous in vivo measurement, the percutaneous microneedle sensor signal conduction does not depend on batch processing, such as washing steps or adding exogenous reagents. In addition, since the percutaneous microneedle sensor signal is generated by a specific, binding-induced conformational change, instead of the target being adsorbed to the sensor surface (in the case of SPR, QCM, FET and microcantilever), the platform is relatively not fouling sensitive. For example, previous studies have shown that the percutaneous microneedle sensor performs well within a few hours in flowing, undiluted serum, making it one of the single-step biosensor platforms with the strongest pollution resistance reported to date.

The percutaneous microneedle array sensing unit of this case is composed of a plurality of protruding metal sheets, at least one of which is a working electrode, and the inner surface of the metal sheet is modified with a sensing polymer. The polymer is an aptamer specific to the target drug molecule. One end is modified with a self-assembled monolayer (SAM), which can be fixed on the inner surface of the working electrode, and the other end is modified with a redox reporter molecule. More specifically, as an example, the working electrode is first coated with gold, and then are modified by various sensing polymers. The sensing polymer is modified with thiol SH at one end, and then an aptamer. The end of the aptamer is modified by Methyl blue. With square wave voltammetry (SWV) or DPV or chronoamperometry, it can continuously detect various inflammation, immune response molecules, or drugs or medicines that can appear in the tissue fluid.

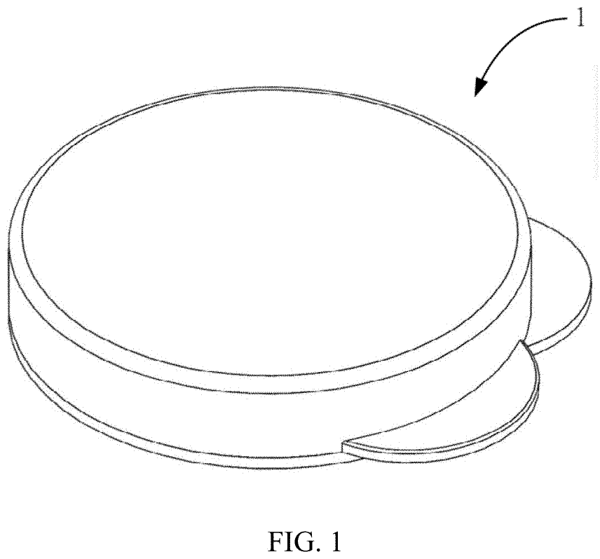

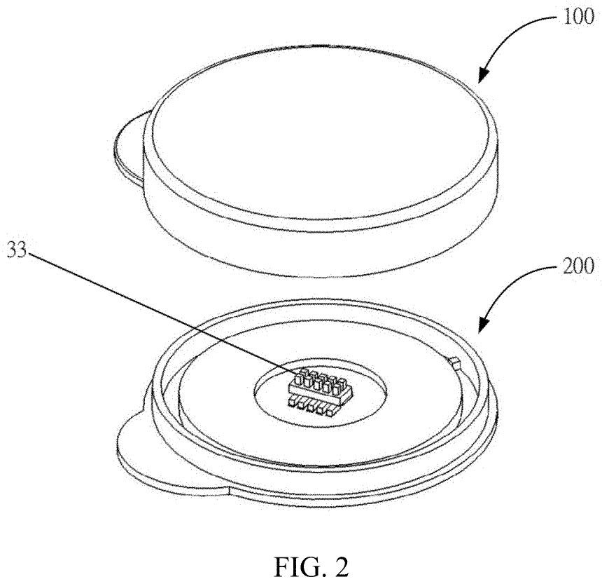

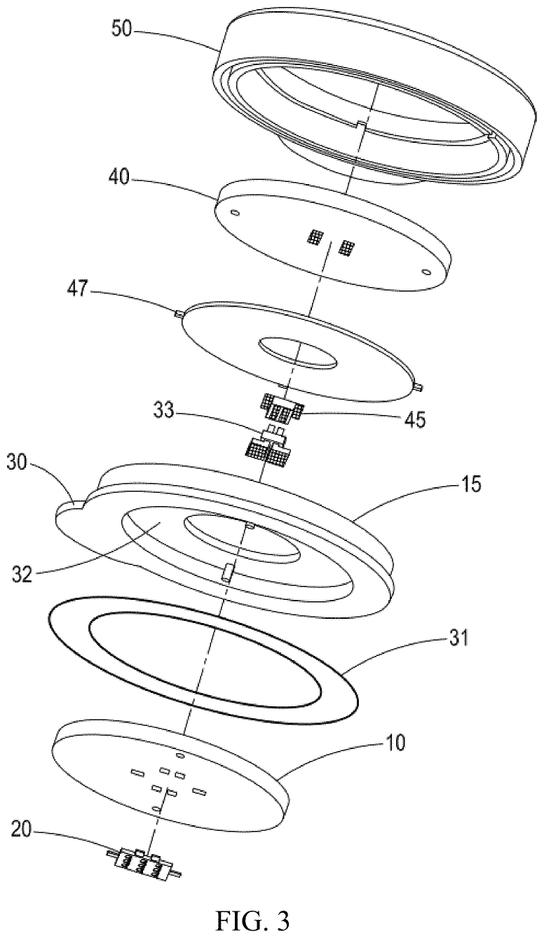

Please refer to FIG. 1, FIG. 2, and FIG. 3. FIG. 1 is an external view of a transdermal microneedle drug monitoring system according to an embodiment of the present invention, and FIG. 3 is an exploded view of the percutaneous microneedle drug monitoring system according to an embodiment of the present invention.

The percutaneous microneedle drug monitoring system 1 of the present invention includes a signal processing device 100 and a microneedle device 200. Please refer to FIG. 3. The microneedle device 200 includes a substrate 10, a base 15, a microneedle unit 20, a flexible adhesive cloth 30 and a release paper 31, and the male connector 33. The microneedle unit 20 and the male connector 33 are arranged on the substrate 10, and the substrate 10 is embedded in the base 15.

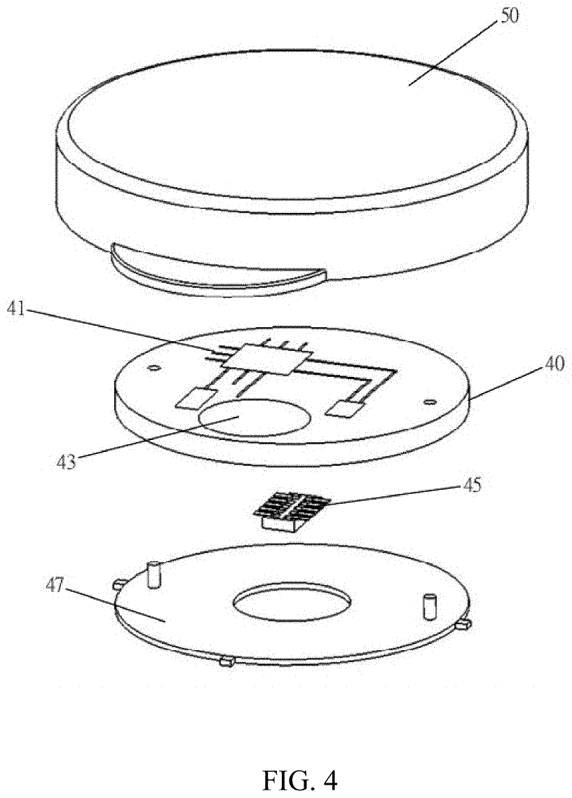

Please refer to FIG. 4, the signal processing device 100 includes a signal processing unit 41, a power supply unit 43, a female connector 45, a cover 47, and an outer cover 50. The signal processing unit 41, power supply unit 43, and female connector 45 are arranged on the circuit board 40. The electrical connection between the signal processing device 100 and the microneedle device 200 is achieved through connectors. The connector of the microneedle device 200 is a male connector 33, and the connector of the signal processing device 100 is a female connector 45, and vice versa. The signal processing device 100 and the microneedle device 200 are mechanically connected by the outer cover 50 and the base 15.

The signal processing unit 41 is electrically connected to the microneedle unit 20 to receive the analyte concentration sensed by the microneedle, and after calculation and determination, the information is converted into a sensing signal, which is transmitted to the user's handheld device via wireless communication, and is a signal that can reflect the user's current physiological state. Common communication specifications such as Bluetooth, RFID, WIFI, LPWA, etc. can be used for wireless communication. The power supply unit 43 supplies working power to the percutaneous microneedle drug monitoring system of the present invention.

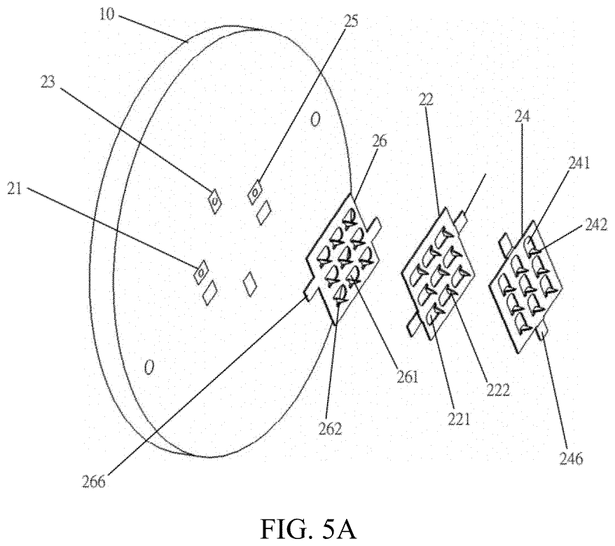

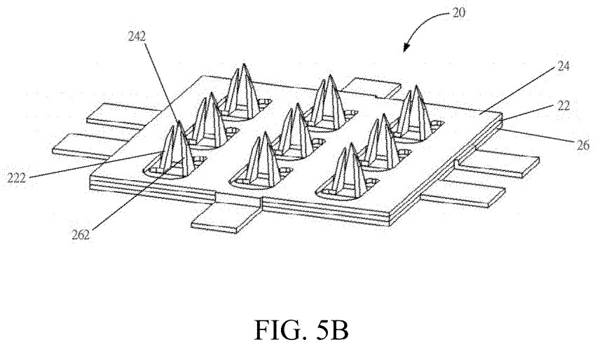

Referring to FIGS. 5A and 5B, according to an embodiment of the present invention, the microneedle unit 20 includes a microneedle unit 20 arranged on a substrate 10; the microneedle unit 20 is composed of a first sheet 22 as a working electrode, the second sheet 24 of the reference electrode and the third sheet 26 as the counter electrode are superimposed on each other, but the three are electrically insulated from each other. The flexible adhesive cloth 30 and the release paper 31 have an opening 32 for the micro-needle unit 20 to pass through, and the micro-needle unit 20 is connected with a male connector on the circuit board 40 through vias 21, 23, 25 45 electrical contacts are electrically connected. Since the present invention has the flexible viscose cloth 30, it can conform to the user's muscle contour and closely contact with the user's skin during operation.

The embodiments of the present invention, as shown in FIGS. 5A and 5B, further illustrate the structure of the microneedles, which are formed by combining a plurality of metal sheets with protruding microneedle arrays on a substrate, and the metal sheets are respectively working electrode, counter electrode, and reference electrode; working electrode, counter electrode, and reference electrode can be made of 0.06-0.1 mm stainless steel sheet, and the height of the protruding array microneedle is 0.6-1.5 mm. In combination, the first embodiment is three-stacks in one (outermost layer: reference electrode; middle layer: working electrode; bottommost layer: counter electrode), and the stacking sequence can be selected arbitrarily. The advantage is that the area is reduced, and the electrically insulating layer can be covered between the layers. In addition, the number of microneedles can be reduced to only 2×2, and the corresponding area is reduced to 2 mm*2 mm.

FIGS. 5A and 5B show an embodiment of the present invention. The microneedle unit 20 is composed of a first sheet 22 (working electrode), a third sheet 26 (counter electrode), and a second sheet 24 (reference electrode). The first sheet is electrically insulated from each other. At least one first perforation 221 is provided on the first sheet, a first spur 222 is provided on the edge of the first perforation, and at least one third perforation 261 is provided on the third sheet 26. The edge of the three perforations 261 is provided with a third spur 262, and the second sheet 24 is provided with at least one second perforation 241, the edge of the second perforation 241 is provided with a second spur 242, the third spur 262 and the second spur 242 passing through the first perforation 221 of the first sheet are formed with the first protruding spur 222 in a triangular pyramid shape or a quadrangular pyramid with a missing side that does not contact each other, so that the subcutaneous tissue fluid can effectively enter the inner surface of the protruding spur and bind with the sensing polymer.

Each metal sheet can be packaged to the substrate PCB by means of SMD. SMD can use room temperature/normal temperature/low temperature conductive silver glue bonding, or UV ultraviolet light curing conductive silver glue bonding, or low temperature riveting. When laminating metal sheets, electrical insulation is required between the layers. The preferred order of stacking arrangement in manufacturing is, the outermost layer: the reference electrode; the middle layer: the working electrode; the bottom layer: the counter electrode, because the working electrode is in addition to covering the sensing polymer, it is usually necessary to cover most of the outermost layer with a porous electrically insulating film. Therefore, if it is placed in the middle layer, it can just electrically isolate the upper reference electrode and the bottom counter electrode. As a result, the reference electrode on the upper layer and the counter electrode on the bottom layer do not need to be covered with an electrically insulating film on the outermost layer in manufacturing.

In assembly, the metal sheet can also be made into the form of DIP, passing through the PCB substrate, and the back side is still combined with room temperature/normal temperature/low temperature conductive silver glue, or UV ultraviolet light curing conductive silver glue. For the purpose of fixation, the substrate adopts a PCB double-layer board, so the assembly can be completed more easily. In order to take into account the biocompatibility, the PCB needs to use a lead-free process. Biocompatible plastic substrates can also be used for production by injection molding.

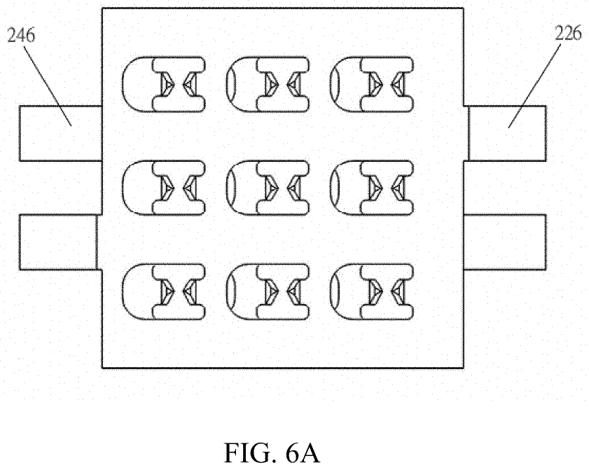

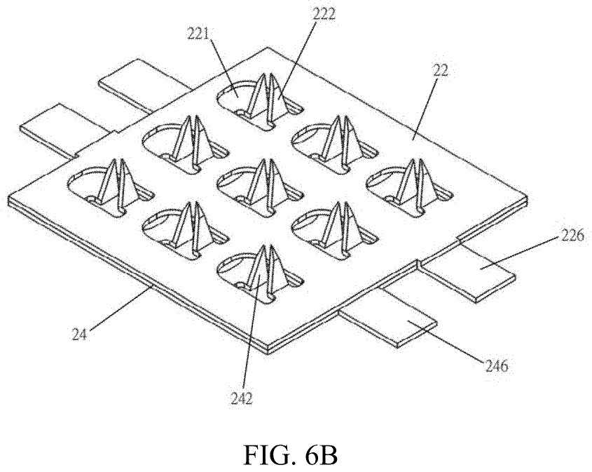

Please refer to FIGS. 6A and 6B. FIGS. 6A and 6B are schematic diagrams of a microneedle unit according to an embodiment of the present invention. The microneedle unit 20 is formed by superimposing a first sheet 22 and a second sheet 24. The first sheet 22 is provided with at least one first perforation 221. The edge of the first perforation 221 is provided with a first spur 222. The second sheet 24 is provided with at least one second perforation 241. The edge of the second perforation 241 is provided with a second spur 242. The second spur 242 passes through the first perforation 221 at the opposite position on the first sheet 22 to face the first spur 222. In addition, an extension pin 226 may be provided on the edge of the first sheet 22 to connect with the solder pad on the substrate 10. An extension pin 246 may be provided on the edge of the second sheet 24 to be connected to the solder pad on the substrate 10.

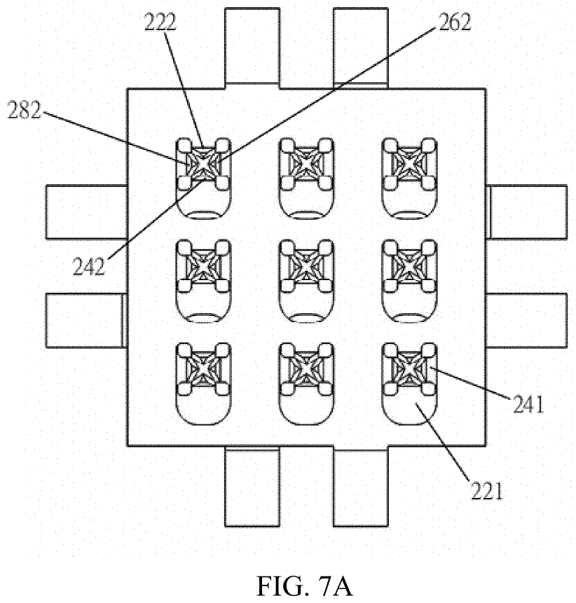

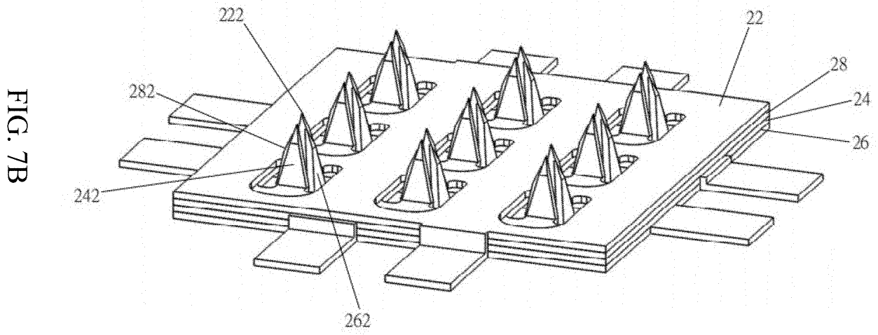

FIGS. 7A and 7B are a partial top view and a combined perspective view of the structure of a microneedle unit according to another embodiment of the present invention. The outermost layer one and two: working electrode; middle layer: counter electrode; bottom layer: reference electrode, the same, the order of stacking can be selected arbitrarily, the microneedle needs to enter the subcutaneous area, still maintain 2 mm*2 mm. This structure can be used to make a two-in-one sensing system, such as measuring blood glucose and insulin at the same time; or measuring endotoxin and antibiotics at the same time.

As shown in FIGS. 7A and 7B, the microneedle unit 20 is composed of a first sheet 22 (first working electrode), a second sheet 24 (second working electrode), a third sheet 26 (counter electrode), and a fourth sheets 28 (reference electrodes) are superimposed, but are electrically insulated from each other. At least one first perforation 221 is provided on the first sheet 22, a first spur 222 is provided on the edge of the first perforation 221; and at least a second perforation 241 is provided on the second sheet 24, a second spur 242 is provided on the edge of the second perforation 241; at least a third perforation 261 is provided on the third sheet 26, a third spur 262 is provided on the edge of the third perforation 261; and at least a fourth perforation 281 is provided on a fourth sheet 28, and a fourth spur 282 is provided on the edge of the fourth perforation 281. The second spur 242, the third spur 262 and the fourth spur 282 pass through the first perforation 221 to form a quadrangular pyramid shape with the first spur 222 on the first sheet 22.

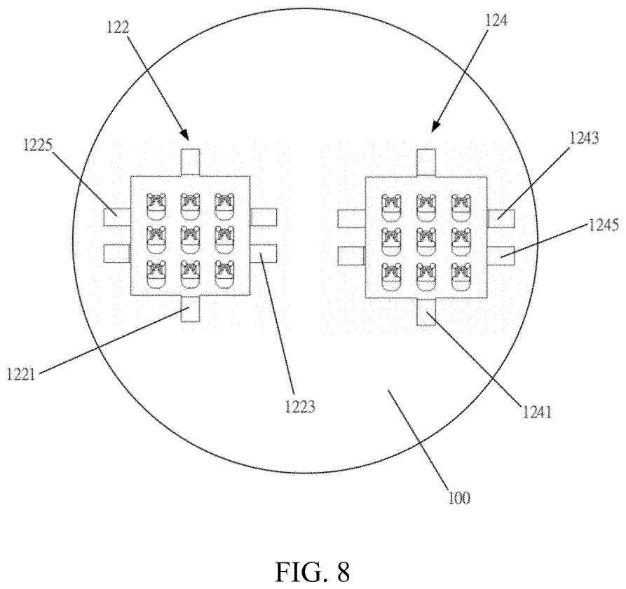

Referring to FIG. 8, there are a first microneedle group 122 and a second microneedle group 124. The first microneedle group 122 is composed of three layers of sheets, which are the first sheet 1221, the second sheet 1223, and the third sheet 1225. The second microneedle group 124 is composed of three layers of sheets, the first sheet 1241, the second sheet 1243, and the third sheet 1245, which are fixed on the substrate 100. Each sheet can have multiple possible applied combinations, as shown in Table 1. This structure can be used to make two sets of two-in-one sensing systems, such as simultaneous measuring two of blood glucose, insulin, PCT, antibiotics; or a three-in-one sensing system, simultaneous measuring three of blood glucose, PCT, lactic acid, antibiotics, etc. It can do four-in-one sensing. For example, simultaneous measuring four of blood glucose, lactic acid, uric acid, IL-6, antibiotics, etc.

TABLE 1

| | 1221 | 1223 | 1225 | 1241 | 1243 | 1245 |
|---|---|---|---|---|---|---|
| 4-in-1 | counter | reference | 1^{st} | 2^{nd} | 3^{rd} | 4^{th} |
| sensor | | | working | working | working | working |
| 3-in-1 | 1^{st} | 1^{st} | 1^{st} | 2^{nd} | 2^{nd} | 3^{rd} |
| sensor | reference | counter | working | counter | working | working |
| 2-in-1 | 1^{st} | 1^{st} | 1^{st} | 2^{nd} | 2^{nd} | 2^{nd} |
| sensor | counter | reference | working | counter | reference | working |

Still referring to FIG. 8, in the 3-in-1 embodiment of the present invention, one of the first microneedle group 122 and the second microneedle group 124 can be the structure of FIG. 7, which is composed of four sheets superimposed to achieve 2-in-1 detection, while the other group still maintains a single detection, so that a 3-in-1 embodiment is possible. In addition, in the 4-in-1 embodiment, these two groups can be configured as shown in FIG. 7 to achieve 2-in-1 detection respectively. Such an embodiment is because the electrochemical detection composed of three electrodes can have enzyme-type sensing polymers, and the main detection circuit is biased towards the electrochemical measurement circuit of the potentiostat; while the use of antibodies, aptamers, or other non-enzyme sensing polymers may prefer to use square wave voltammetry (SWV), differential pulse voltammetry (DPV), or electrochemical impedance spectroscopy (EIS) electrochemical reading circuit. Therefore, to increase the efficiency of reading, it can separate two microneedle groups to correspond to the enzyme and non-enzyme reading circuits. In some embodiments, the reading circuit can be multi-functional, and it has reading circuits for potentiostat, SWV, DPV, and EIS. It only needs to switch in way of software or hardware. In this way, these micro-needles group can use multiplexers in turn to switch to a single electrochemical reading circuit, which can greatly reduce the size of the overall monitoring system, and can simultaneously monitor the concentration of various analytes in the body, which is helpful to the realization of real-time precision medicine.

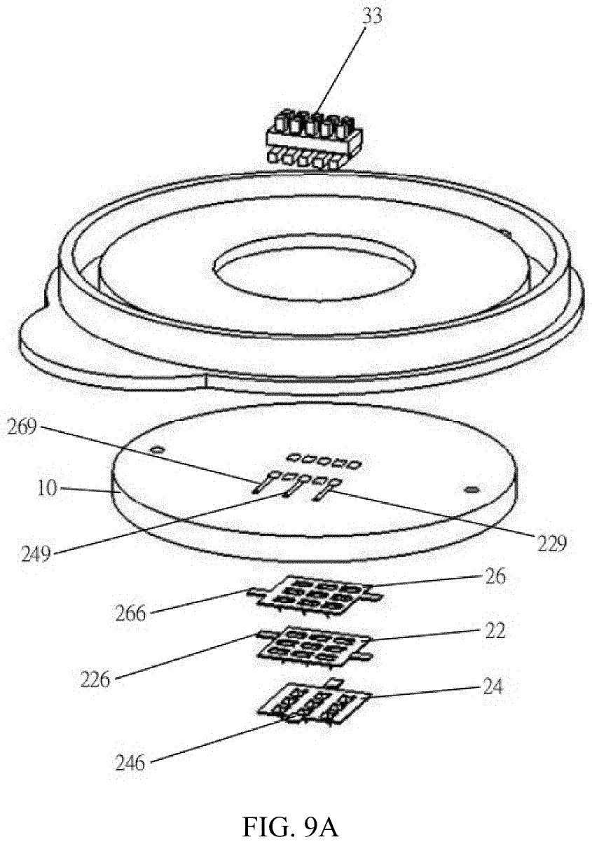

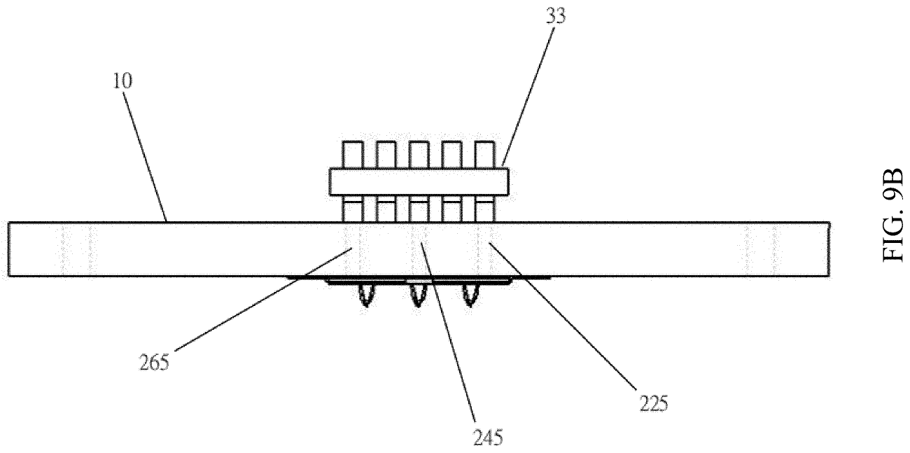

Next, please refer to FIGS. 9A and 9B. FIG. 9A is a schematic diagram of the combination of a percutaneous microneedle drug monitoring system according to an embodiment of the present invention. The microneedle assembly 20 in this embodiment is formed by stacking the first sheet 22, the second sheet 24, and the third sheet 26. For example, a punching force can be applied to the first sheet 22, the second sheet 24, and the third sheet 26 along the four sides of the above three sheets to combine the three. Each sheet of the microneedle group has pins extending from its edges, such as the extension pins 226 of the first sheet 22 (working electrode), the extension pins 246 of the second sheet 24 (counter electrode), and the third sheet 26 (Reference electrode) extension pin 266. These extension pins can be fixed to the PCB substrate 10 by low temperature soldering or silver glue bonding. Refer to FIG. 9B through the vias 225, 245, 265 and the opposite circuit (not shown on the figure) laid on the back of the PCB. For connection, the circuit includes an electrochemical sensing and processing circuit, a wireless communication module, a battery, etc., which means that the three electrodes of the present invention can be effectively connected to the electrochemical sensing and processing circuit.

In another embodiment, referring to FIGS. 3 and 9B, a separate circuit board 40 can also be produced, including electrochemical sensing processing circuits, wireless communication modules, batteries, etc., and then allowing electrochemical The three input points of the sensing processing circuit are assembled using female connectors 45 or connected with spring pins and male connectors 33. Connector 33 is electrically connected to solder pads 229, 249, and 269 on the PCB, and then passes through vias 225, 245, and 265. It is in effective electrical contact with the extension legs 226, 246, and 266 of the three electrodes arranged on the back of the PCB.

Basically, in a preferred embodiment, all the sheets of the microneedle set are made of biocompatible or medical stainless steel materials. During manufacturing, only the inner and outer surfaces of the microneedles are plated with gold, and the reference electrode is only the microneedle is coated with a layer of Ag/AgCl, and the working electrode is coated with sensing polymers, such as enzymes, or aptamers, etc. Special attention should be paid to the coating area, which only needs ⅓ upper or ½ upper of the microneedle height. In addition, insulating parts and preventing biological interference need to be covered with porous materials. These porous materials can be hydrogel or HEMA, epoxy-polyurethane formic resin (Epoxy-PU) membrane, semi-permeable membrane, or low oxygen permeable membrane when the sensing polymer is enzyme. When the sensing polymer is aptamer, it may be polysulfone membrane or the like. In a preferred embodiment, the porous material may be covered the outmost layer of the working electrode, counter electrode and reference electrode to absorb the tissue fluid to contact the microneedles if the height of the microneedle is not enough to immerse all the microneedles directly in the tissue fluid percutaneously.

In a preferred manufacturing embodiment of the microneedle working electrode of the present invention, the protruding array microneedle working electrode is first roughened to increase its active area, and then gold-plated to become a gold electrode. The DNA construct with specificity to the target drug is then reduced with a 1000-fold molar excess of tris(2-carboxyethyl) phosphine for 1 hour at room temperature. Then the freshly roughened gold electrode was rinsed in deionized water, and then immersed in a solution of 200-500 nM of an appropriately reduced DNA construct at room temperature for 1 hour. After that, the microneedle working electrode was covered with polysulfone fiber membrane. The microneedle working electrode is soaked in a 20 mM 6-mercapto-1-hexanol PBS solution at 4° C. overnight for 12 hours to cover the remaining gold surface and remove non-specifically adsorbed DNA. After that, the microneedle working electrode is rinsed with deionized water and stored in PBS.

In one embodiment of the present invention, the microneedles of the microneedle unit 20 are formed by a stamping or etching or electroforming process. The material of the spurs is selected from stainless steel, nickel, nickel alloy, titanium, titanium alloy or silicon material. The spur material can also be resin such as polycarbonate, polymethacrylic acid copolymer, ethylene/vinyl acetate copolymer, Teflon or polyester, and be deposited with biocompatible metal on the surface. The height of the spurs is 400-1500 microns, and the width of the substrate is 200-350 microns. The distance between the tips of these spurs is 500-2000 microns.

### Example 1 (Continuous Measurement of Blood Glucose, Lactic Acid, and Uric Acid, Three-in-One Sensor)

(1) The embodiment of the present invention can refer to FIGS. 5A and 5B to make the sensing microneedle unit. The method of fixing the enzyme used for the detection of metabolites is polymer coating method, and electroplating conductive polymer on the first layer of the working electrode to increase the adhesion of the enzyme and signal response. The second layer is quantitatively coated with enzymes on the inner surface of the microneedles of the working electrode, such as enzymes that can produce extremely high specificity for glucose, lactic acid and uric acid. The third layer is coated on the electrode with the diluted Nafion solution by immersion or quantitative dripping method. The purpose is to modify the electrode to avoid interference factors and improve the sensitivity of the electrode. The fourth layer uses to formulate the porous structure of the polymer film. The purpose is to isolate the external factors that may degrade the enzyme activity to continue the stability of the sensing electrode and the long-term effective response.

(2) The electrochemical working principle of this embodiment is a biosensor that uses electrochemical reaction current to detect the concentration of glucose, lactic acid and uric acid. In the first reaction step, the oxidation state of glucose and enzyme GOD is converted into the oxidation state of gluconic acid, lactic acid and enzyme LOX is converted into pyruvate, and the oxidation state of uric acid and enzyme UOX is converted into urine, GOD, LOX and UOX are reduced at the same time;

(3) The reduced enzymes GOD, LOX and UOX will react with the chemical mediator in another oxidation state to regenerate, and the regenerated enzymes can react with glucose, uric acid and lactic acid. As for the reduced meson, it will oxidize on the electrode to generate electric ions to obtain a current signal. At the same time, the oxidized meson can interact with the reduced enzyme. The electrochemical reading circuit here can choose a three-electrode potentiostat, and the reference voltage is about 0.2-0.6V.

### Example 2 (Continuous Measurement of Keto Acid and Blood Glucose, Two-in-One Sensor)

(1) Refer to the microneedle set in FIG. 7, where the first working electrode and the second working electrode are respectively coated with keto acid enzyme 3-hydroxybutyrate dehydrogenase (3HBDH, EC1.1.1.30), and blood glucose oxidase GOD, and the rest are counter electrodes and reference electrodes

(2) The American Diabetes Association recommends that a blood ketone test that quantifies 3-β-hydroxybutyrate (3HB) is ideal for the diagnosis and monitoring of ketoacidosis managed by diabetic patients. Blood ketones refer to 3-β-hydroxybutyrate (3HB), acetone acetate (AcAc) and acetone. These three ketone bodies are produced by the liver and are used as energy sources when glucose cannot adequately provide energy for somatic cells. 3HB and AcAc are the main ketone compounds in human subjects, and the concentration level of acetone in the blood is relatively low. For a normal person, the ratio between 3HB and AcAc is about 1:1, and under DKA (diabetic ketoacidosis, an important symptom related to diabetes), the ratio may be as high as 10:1. Therefore, the detection of 3HA is recommended for the management of DKA.

(3) Because patients with type 1 diabetes can take oral medications, they may cause ketoacidosis. In addition, taking food, because of fear obesity, avoiding carbohydrates, may also cause ketoacidosis. Ketoacidosis usually has no discomfort. When discomfort occurs, the ketoacid may be seriously exceeded. Therefore, the two-in-one sensor of present invention that detects keto acid and blood sugar at the same time has its value.

(4) The electrochemical sensing of keto acid is mainly to monitor the concentration of ketone 3-β-hydroxybutyric acid (3HB) in physiological fluid in the management of potential diabetic patients. The current electrochemical detection of 3HB involves at least two stepwise reactions, which may also require a mediator to facilitate electron transfer. The detection method in this embodiment only involves one reaction step and does not require any mediator.

(5) The biosensor operates at a relatively low electrochemical potential (relative to Ag/AgCl+200 mV), and fixes the enzyme 3-hydroxybutyrate dehydrogenase (3HBDH, EC1.1.1.30) on the working electrode (micro-needle tip) modified with conductive polymer, NADH (nicotinamide adenine dinucleotide, reduced form) is detected, which is the reaction product of 3HB and NAD+(nicotinamide adenine dinucleotide, oxidized) in the presence of 3HBDH. Electrochemical measurements show that this biosensor responds well to 3HB in phosphate buffer and 100% bovine serum. The reaction of 3HB and NAD+(nicotinamide adenine dinucleotide, oxidized form) is catalyzed by the enzyme 3-hydroxybutyrate dehydrogenase (3HBDH, EC 1.1.1.30) to produce AcAc (acetone acetate) and NADH (nicotinamide adenine dinucleotide, reduced form) is shown in reaction (1).

$$3 \text{-} β \text{-} Hydroxybutyrate + {NAD}^{+} {\to }^{3 HBDH} AcAc + NADH + {H}^{+} \qquad ( 1 )$$

(6) The manufacturing process of this three-electrode configuration enzyme sensor is described as follows:

(a) Preparation of ink-based solutions: The ink-based solution for printing the working electrode is prepared by mixing phosphate buffer, enzyme fixative and thickening polymer. Usually 10 ml of pH 7.0 phosphate buffer is mixed with 1.36 ml of polyethyleneimine and 0.34 g of 2-hydroxyethylcellulose to obtain an ink-based solution for printing working electrodes. The mixing is complete when a clear homogeneous solution is obtained.

(b) Preparation of AgCl/Ag reference electrode: For the reference electrode, a thick AgCl/Ag film is used and printed on the stainless steel electrode to serve as the Ag/AgCl reference electrode.

(c) Preparation of Enzyme 3HBDH ink: Enzyme 3HBDH ink was prepared in the following manner and applied to the sensor prototype. Mix 1 ml of ink from step (a) with 125 units of enzyme 3HBDH, 150 mg NAD+ and 5.0 mg bovine serum albumin. The mixing of these components is carried out until a clear solution is obtained.

(d) Manufacturing of biological sensors: Then, the enzyme ink from step (c) is coated on the working electrode to form the working electrode of the biosensor.

### Example 3 (Three-in-One Sensor: Keto Acid, Insulin, Blood Sugar)

(1) The fabrication of the microneedle working electrode for keto acid and blood glucose is as shown in the first embodiment. It can refer to the microneedle structure in FIG. 8, which will not be repeated here.

(2) Insulin is a hormone composed of a double-stranded polypeptide with a molecular weight of 5808 Da. It is produced by pancreatic beta cells to keep blood glucose levels from being too high (hyperglycemia) or too low (hypoglycemia). The insulin concentration in the blood of diabetic patients is lower than normal (57-79 picomoles).

(3) Currently, there are a variety of analytical methods used for insulin detection, such as high performance liquid chromatography (HPLC), ultraviolet-visible detector, liquid chromatography-tandem mass spectrometry (LC-MS), capillary electrophoresis, surface plasmon resonance (SPR), fluorescence spectroscopy and electrochemical biosensors. Among them, electrochemical biosensors are highly sensitive, selective and cost-effective methods. Electrochemical immunosensors and aptamer sensors are the two main types of electrochemical biosensors that have been proposed for the determination of biologically important compounds. However, electrochemical immunosensors have some disadvantages, such as expensive manufacturing processes and instability of antibodies

The present invention uses a microneedle working electrode to connect aptamers to detect the concentration of insulin in the subcutaneous tissue fluid in real time. The aptamers that can be selected are as follows:

5′-HS-(CH2)6-GGTGGTGGGGGGGGTTGGTA GGGTGTCTTC-(CH2)
2-MB-3′

In the present invention, as the concentration of blood glucose in the subcutaneous tissue fluid is 80-90% of the blood concentration in the blood vessel, a calibration model needs to be established to achieve an error of ±15%-20%. In one embodiment of the present invention, the insulin concentration reading circuit can use square wave voltammetry (SWV), and it needs to use a dual-frequency method for detection, which can avoid differences in individual sensing microneedles such as aptamer coating.

### Embodiment 4: Doxorubicin

(1) Doxorubicin is a bacterial antibiotic widely used in the treatment of leukemia and various other cancers, and its blood concentration needs to be monitored when used. Refer to the microneedle set in FIGS. 5A and 5B. The working electrode needs to be fixed with Doxorubicin DNA construct, and the rest are counter electrodes and reference electrodes.

(2) Methylene blue and thiol modified Doxorubicin DNA construct. The 5′end of each is modified with a thiol on the 6-carbon linker, and the 3′end is modified with a methylene blue modified linker that is linked to DNA by forming an amide bond with a primary amine on the 7-carbon, as follows:

5′-HS-(CH2)6-ACCATC TGTGTAAGGGGTAAGGGGTGGT-
(CH2)7-NH-Methylene Blue-3′

(3) The length of the surface-bound carbon linker represents a compromise between the two main criteria (stability and electron transfer efficiency) for electrochemical biosensor applications. A 6-carbon linker was chosen here because it exhibits good stability. The construct was dissolved to 200 μM in 1× Tris-EDTA buffer and frozen in separate aliquots at −20° C. until use.

### Embodiment 5: Aminoglycoside

(1) Aminoglycoside antibiotics are a class of antibiotics with the structure of amino sugars and aminocyclines. They are mainly used in the clinical treatment of Gram-negative bacteria, Pseudomonas aeruginosa and other infections, because these drugs often have relatively serious ototoxicity and renal toxicity, its application is subject to certain restrictions, and its blood concentration needs to be monitored during use. Refer to the microneedle set in FIGS. 5A and 5B. The working electrode needs to be fixed with the Aminoglycoside DNA construct, and the rest are counter electrodes and reference electrodes.

(2) Aminoglycoside DNA construct modified with methylene blue and thiol. The 5′ end of each is modified with a thiol on the 6-carbon linker, and the 3′ end is modified with a methylene blue modified linker that is linked to DNA by forming an amide bond with a primary amine on the 7-carbon, as follows:

5′-HS-(CH2)6-GGGACTTGGTTTAGGTAATGAGTCCC-(CH2)7-
NH-Methylene Blue-3′

(3) The length of the surface-bound carbon linker represents a compromise between the two main criteria (stability and electron transfer efficiency) for electrochemical biosensor applications. A 6-carbon linker was chosen here because it exhibits good stability. The construct was dissolved to 200 μM in 1× Tris-EDTA buffer and frozen in separate aliquots at −20° C. until use.

In one embodiment of the present invention, the microneedle working electrode is used to connect aptamer to detect the analyte specifically detected by the aptamer contained in the subcutaneous tissue fluid in real time, and the reading circuit can also use chronoamperometry, which differs from SWV in that SWV converts changes in electron transfer rate into changes in peak current, thereby indirectly reporting its transfer kinetics, while chronoamperometry directly measures electron transfer kinetics. It responds to increasing the potential of the electrode to the value at which the redox reporter is completely oxidized or completely reduced by determining the lifetime of the current transient.

For example, when the sensor of the present invention is sensitive to the reaction of aminoglycoside antibiotics to a sufficiently negative potential, the resulting current decay curve is a multi-exponential phase. Specifically, if there is no target drug molecule in the sample, they show a fast exponential phase, their lifespan is 100±3 (the error represents the standard error derived from 5 independently manufactured electrodes), and they have 6.5±0.5 ms slower phase of life. We attribute the faster phase to the double-layer charge formed on the electrode surface under this potential bias (i.e., the migration of water-soluble ions, with a time scale of 18 microseconds), which does not change the target drug concentration. On the contrary, the slower charge transfer rate corresponding to methylene blue is related to the change of target drug concentration. Therefore, after adding saturated target drug molecules, the slower phase becomes faster, with a lifetime of 1.20±0.01 milliseconds.

In one embodiment of the present invention, the reading circuit may use intermittent pulse amperometry (IPA) technology to interrogate the balanced and dynamic target drug molecules and the working electrode surface of the microneedle sensor of the present invention. The combination of aptamers achieves a time resolution of 2 milliseconds. The microneedle sensor of the present invention includes a microneedle working electrode surface modified with a flexible nucleic acid aptamer, which is connected to a redox active molecule at the 3′end. The introduction of the target drug molecule changes the conformation and flexibility of the nucleic acid aptamer, which changes the charge transfer rate of the additional redox molecule (methylene blue). Generally, voltammetry, such as SWV, monitors the change of charge transfer rate in this type of sensor. In this embodiment, the use of IPA can detect the change in the charge transfer rate (i.e., current) within <100 μs after the potential pulse is applied. The change of the sensor IPA current is quantitatively related to the concentration of the target analyte. The greater the change in current, the higher the concentration of the target analyte. In addition, IPA is used to quickly detect electrochemical surfaces with a time resolution equivalent to twice the width of the potential pulse used, which was not possible with the traditional voltammetry techniques (AC, square wave, cycle) previously used. The intermittent pulsed amperometry exhibits an unprecedented sub-microsecond time response and is a general method for measuring the performance of fast sensors.

In one embodiment of the present invention, the reading circuit may use fast-scan cyclic voltammogram (FSCV) technology to interrogate the balance and dynamic target drug molecules and the microneedle sensor of the present invention. The combination of aptamers on the working electrode surface achieves a time resolution of several to tens of milliseconds. For example, the voltage rises with a 10 Hz cyclic triangle wave and then drops to cause oxidation and reduction of the target molecule. Although the background current must be subtracted, the cyclic voltammogram (CV) after the background subtraction helps to identify the concentration of the detected drug molecule. The microneedle sensor of the present invention includes a microneedle working electrode surface modified with a flexible nucleic acid aptamer, which is connected to a redox active molecule at the 3′end. The introduction of the target drug molecule changes the conformation and flexibility of the nucleic acid aptamer, which changes the charge transfer rate of the additional redox molecule (methylene blue). In the present embodiment, using FSCV can detect the change in charge transfer rate (i.e., current) after applying a cyclic triangular wave. The change of the sensor FSCV current is quantitatively related to the target analyte concentration, and the greater the current change, the higher the target analyte concentration.

Due to the use of a specific aptamer to modify the surface of the microneedle working electrode, it is not easy to maintain the individual difference of each sensor within an acceptable range in manufacturing. Therefore, if it is equipped with an electrochemical reading circuit such as chronoamperometry, IPA, etc., because the relative comparison method is used to obtain the concentration, so the difficulty in calibration and the difference in manufacturing can be avoided.

The signal processing embodiment of the present invention can also use electrochemical impedance spectroscopy (EIS), because EIS may be able to detect molecular concentrations as low as pg/mL. However, EIS is a quasi-static measurement method, which does not use a redox method, and cannot measure the concentration of target drug molecules in transdermal tissue fluid in real time or continuously.

One way to solve the above-mentioned problems may be to use a redox reporter molecule to modify the aptamer so that it can reversely restore the aptamer bound to the target drug molecule. In other words, the above-mentioned real-time measurement methods such as SWV, chronoamperometry, IPA or FSCV are generally not sensitive enough to detect the concentration of pg/mL, but they can restore the binding between the aptamer and the target drug molecule. Therefore, it can combine continuous real-time methods and static methods. That is to combine SWV, chronoamperometry, IPA or FSCV with EIS. First use the continuous real-time method for measurement. When the steady state is reached, it can be switched to EIS for high-sensitivity measurement. Or use EIS to measure first, and then switch to the redox method, so that the aptamer can be separated from the target molecule, and the aptamer can be restored to its empty state, which is convenient for repeated use of EIS to measure.

Here is required to give a special statement that the present invention measures amino acids, sugars, fatty acids, coenzymes, hormones, neurotransmitters, salts and wastes produced by cells, drugs and other analytes in percutaneous tissue fluid and in blood, the corresponding analytes are not equal in concentration, usually several ten times. Therefore, calibration is needed to accurately infer the target analyte concentration in the blood according to the present invention. This part of the technology is compatible with the continuous blood glucose monitoring system (CGMS), it won't repeat them here.

In addition, the sensing microneedle touches the skin and partially invades subcutaneously. During the measurement, the working electrode and the counter electrode or the reference electrode of the conventional microneedle patch are in contact with the skin respectively, resulting in the positive electrode with a certain distance from the negative electrode. When using the potentiostat measurement method, the sweat glands between the positive electrode and the negative electrode produce reverse iontophoresis and ion penetration effects, which stimulate the discharge of sweat and affect the measurement of tissue fluid analysis. In order to reduce this effect, the sensing microneedle overlaps the counter electrode/reference electrode through the working electrode and the counter electrode/reference electrode, so that the distance between the positive electrode and the negative electrode is zero, and the sweat glands can be prevented from producing reverse iontophoresis. Assuming 100 glands/cm2 and 4 nL/min per gland, when the conventional positive and negative electrodes are separated by 0.5 cm, it is equivalent to 0.25 cm² of sweat at the skin-electrode interface to produce 100 nL/min. However, the present invention overlaps the positive and negative electrodes, and the microneedle spacing between the positive and negative electrodes is 0.5 mm, which is equivalent to less than 1 nL/min of sweat produced at the skin-electrode interface of less than 0.0025 cm². Basically, it is less than the amount of sweat that hardly affects the results of the subcutaneous microneedle measurement.

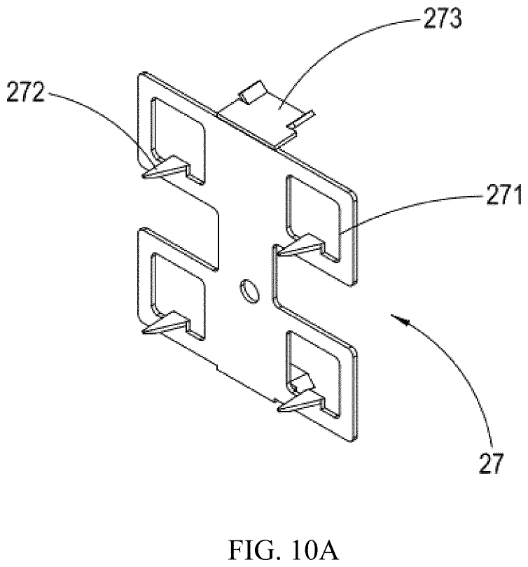

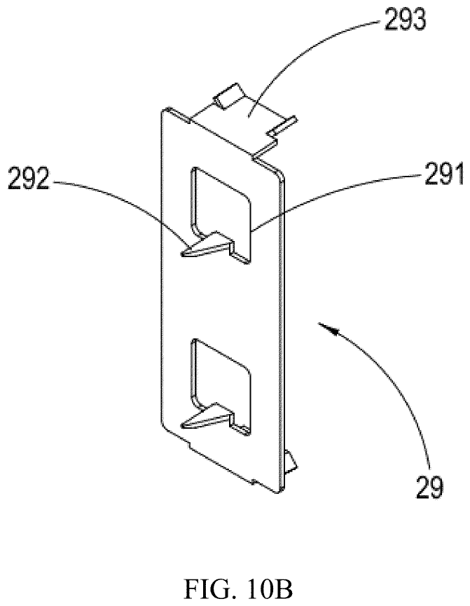

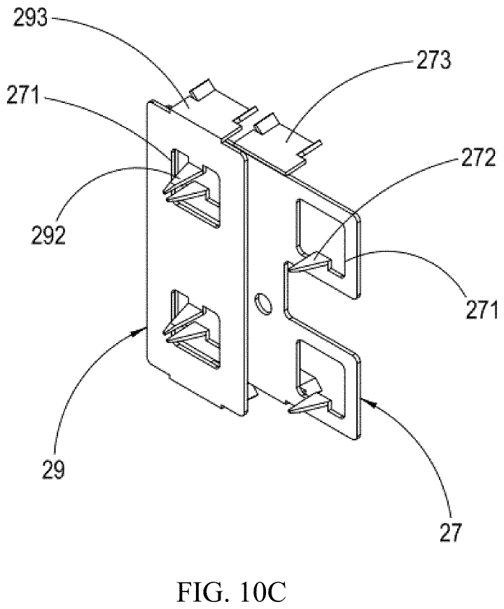

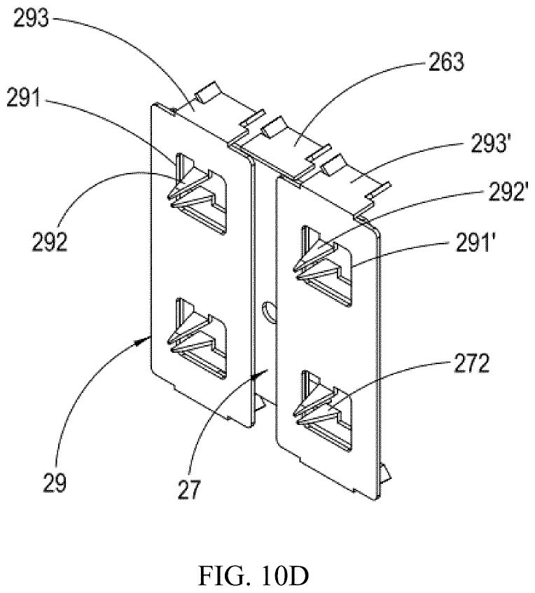

In addition, the present invention can also use the microneedle sheet style shown in FIGS. 10A to 10C, as described below:

It can be seen from FIG. 10A that the microneedle sheet 27 of the working electrode has four perforations 271, four sensing microneedles 272 and two connecting ends 273;

It can be seen from FIG. 10B that the microneedle sheet 29 of the reference electrode (which can also be used as a counter electrode) has two perforations 291, two sensing microneedles 292, and two connecting ends 293;

As shown in FIG. 10C, the microneedle sheet 27 and the microneedle sheet 29 can be superimposed on each other for measurement, which can be used with different microneedle sheets 29 or stacked microneedle sheets 29 to achieve three-in-one or even four-in-one biochemical measurement.

As shown in FIG. 10D, the working electrode microneedle sheet 27 can be overlapped with the reference electrode microneedle sheet 29, and the counter electrode microneedle sheet 29′ (with two perforations 291′, two sensing microneedles 292′ and two connecting ends 293′) are also stacked on the other side of the working electrode microneedle sheet 27, but are not in contact with the reference electrode microneedle sheet 29, the three-electrode electrochemical measurement can be performed.

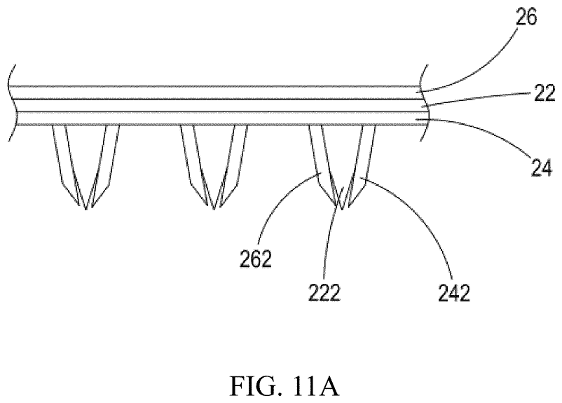

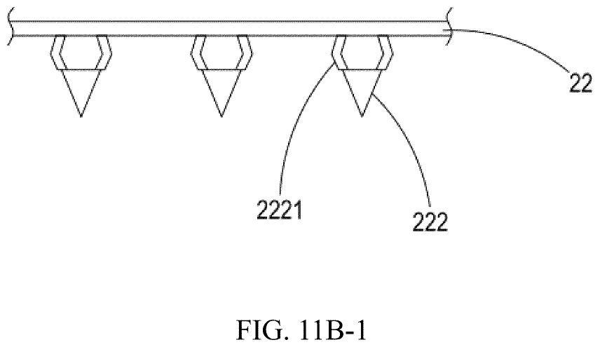

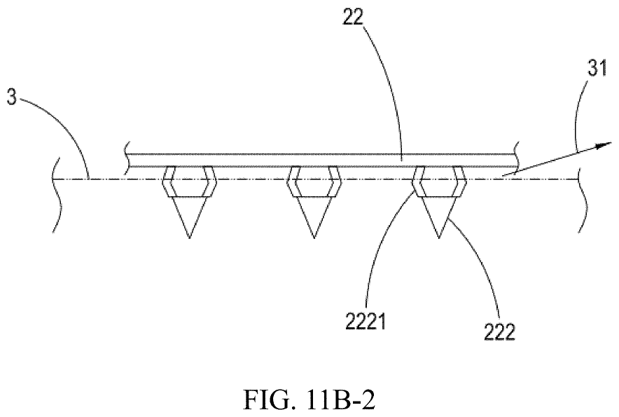

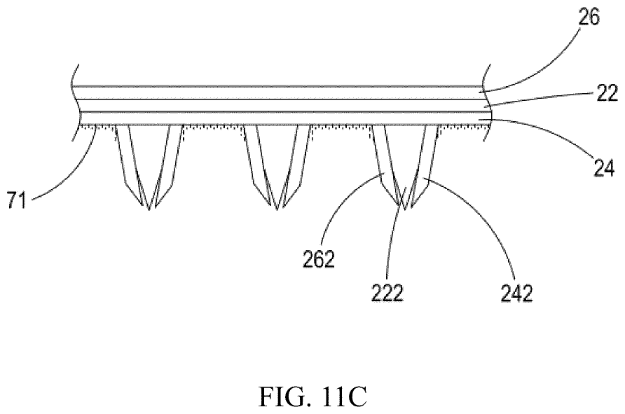

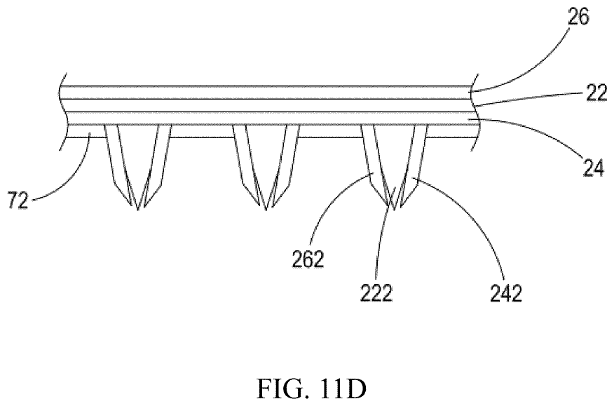

In addition, if it is applied to the motion sensing of athletes, a large amount of sweat will be produced due to exercise. If only the sensing microneedle shown in FIG. 11A is used, it is often because the human body sweat 61 of the subcutaneous tissue 6 will contact the spurs. As shown in FIGS. 11B-1 and 11B-2, a sweat blocking element (protruding part 2221) is designed at the bottom and tail of the first spur 222 as shown in FIGS. 11B-1 and 11B-2. The human body sweat generated around the bottom of the first spur 222 cannot touch the tip of the first spur 222, so the interference factor of the sweat 61 to the tip sensing of the first spur 222 can be eliminated.

As shown in FIG. 11C, the bottom of the thorns 222, 242, 262 can also be covered with a non-porous polymer layer 24 (or the non-porous polymer layer 24 is covered first and then the sweat inhibitor (not shown in the figure)), such as aluminum chloride (ACH); aluminum chloride hexahydrate cream, such as Drysol; or anticholinergic medications, such as glycopyrrolate. This makes most of the sweat glands in the part around the bottom of the sensing microneedle 212, 222, 232 that are in contact with the skin be temporarily blocked by the antiperspirant and not sweat, so the sweat will not touch the tip of the protruding spur 222, 242, 262, to eliminate the interference factors of sweat on the tip sensing of spikes 222,242,262.

As shown in FIG. 11D, a sweat blocking element (adsorption structure 72) can also be designed around the bottom of the spurs 222, 242, 262, so that the sweat produced around the bottom of the spurs 222, 242, 262 can be absorbed thus the interference factor of the sweat on the tip sensing of the spikes 222, 242, 262 is eliminated. In addition, the adsorption structure 72 used in this case can be made of a polymer material such as hydrogel or a filter material made of a glass fiber material.

In addition, the bottom of the spurs 222, 242, 262 can also be coated with a sweat blocking element (non-porous polymer material) so that animal body sweat produced around the bottom of the spurs 222, 242, 262 cannot invade the tips of the spurs 222, 242, 262, It is used to eliminate the interference factors of animal sweat on the tip sensing of spikes 222,242,262.

In addition, sweat blocking elements (ditch structure (not shown in the figure)) can also be designed around the bottom of the spurs 222, 242, 262, so that the body sweat produced around the bottom of the spurs 222, 242, 262 can be guided to outsides of the spurs 222, 242, and 262 to volatilize externally to eliminate the interference factor of the body sweat on the tip sensing of the spurs 222, 242, and 262.

In addition, the bottom of the sensing microneedle can also be coated with a non-porous polymer layer and then coated with a perspiration inhibitor, such as aluminum chloride (ACH), aluminum chloride hexahydrate cream, such as Drysol or anticholinergic medications, such as glycopyrrolate, which makes most of the sweat glands on the part of the microneedle substrate in contact with the skin temporarily be prevented from sweating, so it will not affect the sensing of the microneedle.

When compared with other conventional technologies, the percutaneous microneedle monitoring system provided by the present invention has the following advantages:

The microneedles of the microneedle set of the present invention are formed by a stamping or etching or electroforming process and have sufficient mechanical strength. When the microneedles of the microneedle set penetrate the skin for sensing, the microneedles can remain intact.

The structure of the working electrode microneedle assembly of the present invention is advantageous for coating the sensing polymer on the inner surface of the tip of the microneedle. When the microneedles of the working electrode microneedle assembly pierce the skin for sensing, the sensing polymer can be reduced of peeling.

The present invention can use the percutaneous microneedle array to measure the analytes contained in the interstitial fluid (ISF) of the dermis and measure the concentration of the target drug molecules under the skin to learn the medication compliance and body pharmacokinetics.

The present invention has been disclosed above through the above-mentioned embodiments, but it is not intended to limit the present invention. Anyone familiar with this technical field with ordinary knowledge should understand the aforementioned technical features and embodiments of the present invention without departing from the scope of the present invention. Within the spirit and scope, some changes and modifications can be made. Therefore, the patent protection scope of the present invention shall be subject to the definition of the claims attached to this specification.

In summary, the present invention uses a wearable device to pass a signal transceiver in a race path, and the signal transceiver receives the time synchronization control signal of the gateway, which significantly reduces the inaccurate timing of the race due to time synchronization, inaccurate position analysis calculations, and delays in accident rescue.

Although the present invention has been disclosed as above with the examples, it is not intended to limit the present invention. Any person with ordinary knowledge in the technical field can make some modifications and retouching without departing from the spirit and scope of the present invention. The protection scope of the present invention shall be determined by the scope of the attached patent application.

What is claimed is:

1. A percutaneous microneedle monitoring system, comprising:
a substrate;
a microneedle unit includes at least a first microneedle group and a second microneedle group arranged on the substrate, the first microneedle group serves as a working electrode, the second microneedle group serves as a reference electrode, and each microneedle group includes at least one microneedle and is a thin sheet, the first microneedle group and the second microneedle group overlap each other but are electrically insulated from each other, wherein each sheet is provided with at least one perforation, and the edge of the perforation is provided with a spur, wherein the perforations on one sheet allow the spurs at the edges of the perforations at corresponding positions on the remaining sheets to pass through, and the spurs are separated from each other;
a signal processing unit, which is arranged on the substrate and electrically connected to the first microneedle group and the second microneedle group; and
a power supply unit supplies working power to the monitoring system.

2. The percutaneous microneedle monitoring system according to claim 1, wherein a thin sheet of the first microneedle group is stacking with a thin sheet of a second microneedle group, but is electrically insulated from each other, and at least one first perforation is provided on the sheet of the first microneedle group, the first perforation edge is provided with a first spur, and the sheet of the second microneedle group are provided with at least one second perforation, and the second perforation edge is provided with a second spur, the second spur passes through the first perforation at the opposite position on the sheet of the first microneedle group and is opposite to the first spur.

3. The percutaneous microneedle monitoring system according to claim 2, wherein the microneedle unit further includes a third microneedle group as a counter electrode, and the first microneedle group is composed of a first sheet and a second microneedle group are formed by superimposing the second sheet and the third microneedle group by the third sheet, but are electrically insulated from each other. at least one first perforation is provided on the first sheet, and a first perforation is provided on the edge of the first perforation. the second sheet is provided with at least one second perforation, the second perforation edge is provided with a second protruding thorn, and the third sheet is provided with at least one third perforation, the third perforation edge is provided with a third protruding, the second spur and the third spur pass through the first perforation on the first sheet and the first spur is in a triangular pyramid shape or a quadrangular pyramid with a missing side.

4. The percutaneous microneedle monitoring system according to claim 3, wherein the microneedle unit further includes a fourth microneedle group as the second working electrode, where the first microneedle group consists of a first sheet, the second microneedle group consists of a second sheet, the third microneedle group consists of a third sheet, and the fourth microneedle group consists of a fourth sheet are stacked but electrically insulated from each other, the first sheet is provided with at least one first perforation, the edge of the first perforation is provided with a first spur, the second sheet is provided with at least a second perforation, and the edge of the second perforation is provided with a second spur, the third sheet is provided with at least one third perforation, the edge of the third perforation is provided with a third spur, and the fourth sheet is provided with at least a fourth perforation, and the edge of the fourth perforation is provided with a fourth spurs, the second spur, the third spur and the fourth spur pass through the first perforation on the first sheet and form a quadrangular pyramid with the first spur.

5. The percutaneous microneedle monitoring system according to claim 1, wherein further includes at least one microneedle unit, which can simultaneously sense an increase in the types of analytes or/and drugs under the skin.

6. The percutaneous microneedle monitoring system according to claim 4, wherein the microneedle of the first microneedle group, the second microneedle group, the third microneedle group, and the fourth microneedle group is formed by a stamping or etching or electroforming process.

7. The percutaneous microneedle monitoring system according to claim 1, wherein the signal processing unit is mainly selected from electrochemical sensing circuits, cyclic voltammogram, amperometry, square wave voltammetry (SWV), differential pulse voltammetry (DPV), chronoamperometry, intermittent pulse amperometry (IPA), fast-scan cyclic voltammogram (FSCV), electrochemical impedance spectrum (EIS) or its combination.

8. The percutaneous microneedle monitoring system according to claim 1, wherein the working electrode further includes a porous protective layer formed on the sensing polymer or an anti-skin allergy drug.

9. The percutaneous microneedle monitoring system according to claim 8, wherein the porous protective layer is covered the outmost layer of the working electrode, counter electrode and reference electrode to absorb the tissue fluid to contact the microneedles when the height of the microneedle is not enough to immerse all the microneedles directly in the tissue fluid percutaneously.

10. The percutaneous microneedle monitoring system according to claim 1, wherein the material of the spurs is selected from stainless steel, nickel, nickel alloy, titanium, titanium alloy or silicon material, and is depositing biocompatible metal on the surface; or the material of the spurs is resin, and depositing a biocompatible metal on the surface.

11. The percutaneous microneedle monitoring system according to claim 1, wherein the height of the spurs is 300-3000 microns.

12. The percutaneous microneedle monitoring system according to claim 1, wherein the width of the base of the spurs is 150-450 microns.

13. The percutaneous microneedle monitoring system according to claim 1, wherein the inner surface of the working electrode is modified with a sensing polymer, and the sensing polymer is specific for the target analyte such as antibody, aptamer, recombinant monomers (ScFv), carbohydrates, one end of which is modified with self-assembled monolayer (SAM), which can be fixed on the inner surface of the working electrode.

14. The percutaneous microneedle monitoring system according to claim 13, wherein the sensing polymer is an enzyme specific to the target analyte.

15. The percutaneous microneedle monitoring system according to claim 13, wherein or the sensing polymer is an aptamer specific to the target drug molecule. One end is modified with a SAM, which can be fixed on the inner surface of the working electrode, and the other end is modified with a redox reporter molecule.

16. A percutaneous microneedle monitoring system, comprising:
a signal processing device includes a signal processing unit, a power supply unit, a female connector, a cover plate and an outer cover, wherein the signal processing unit, the power supply unit, and the female connector are arranged on a circuit board; a microneedle device includes a substrate, a base, a microneedle unit, a flexible adhesive cloth, a release paper, and a male connector, wherein the microneedle unit and the male connector are arranged on the substrate, and the substrate is embedded in the base, and the microneedle unit at least includes a first microneedle group, a second microneedle group, and a third microneedle group arranged on the substrate, the first microneedle group as a working electrode, the second microneedle group serves as a reference electrode, the third microneedle group serves as a counter electrode, and the inner surface of the working electrode is modified with a sensing polymer and a porous protective layer; and the electrical connection between the signal processing device and the microneedle device is achieved by a connector, the connector of the microneedle device is a male connector, and the connector of the signal processing device is a female connector, and vice versa; in addition, the mechanical connection between the signal processing device and the microneedle device is achieved by the outer cover and the base.
